SEQUENCE LISTING (1 sequence)

SEQ ID NO: 1 - protein, 8 aa, Artificial Sequence
  Synthetic Protein
SIINFEKL

(19) United States
(12) Patent Application Publication
(10) Pub. No.: US 2013/0259898 A1
(43) Pub. Date: Oct. 3, 2013

(54) Vaccine Composition

(71) Applicants: Megan Macleod, Glasgow (GB); Amy McKee, Denver, CO (US); John W. Kappler, Denver, CO (US); Philippa Marrack, Denver, CO (US)

(72) Inventors: Megan Macleod, Glasgow (GB); Amy McKee, Denver, CO (US); John W. Kappler, Denver, CO (US); Philippa Marrack, Denver, CO (US)

(73) Assignee: NATIONAL JEWISH HEALTH, Denver (CO)

(21) Appl. No.: 13/876,089

(22) PCT Filed: Nov. 9, 2010
(86) PCT No.: PCT/US10/56031

§ 371 Date: Jun. 14, 2013

Related U.S. Application Data
(60) Provisional application No. 61/259,322, filed on Nov. 9, 2009.

(51) Int. Cl. A61K 39/235; A61K 39/015; A61K 39/02; A61K 39/002; A61K 39/12; A61K 39/145

(52) U.S. Cl. CPC A61K 39/235; A61K 39/12; A61K 39/145; A61K 39/02; A61K 39/002; A61K 39/015
USPC 424/209.1; 424/184.1; 424/204.1; 424/233.1; 424/234.1; 424/265.1; 424/272.1

(57) ABSTRACT

Disclosed are compositions and the use of the compositions for protection against pathogens comprising an isolated internal pathogenic protein, a TLR agonist and an aluminum salt.

### CROSS REFERENCE TO RELATED APPLICATIONS

The present application claims the benefit of priority under 35 U.S.C. §119(e) from Provisional Patent Application Ser. No. 61/259,322 filed on Nov. 9, 2009. Provisional Patent Application Ser. No. 61/259,322 is incorporated herein by reference in its entirety.

### GOVERNMENT SUPPORT

This invention was supported in part with funding provided by USAMRAMC Grant No. W81XWH-07-1-0550 awarded by the United States Department of Defense. The government has certain rights to this invention.

### REFERENCE TO A SEQUENCE LISTING

This application contains a Sequence Listing submitted electronically as a text file by EFS-Web. The text file, named “2879-149-PCT_ST25”, has a size in bytes of 1 KB, and was recorded on 9 Nov. 2010. The information contained in the text file is incorporated herein by reference in its entirety pursuant to 37 CFR §1.52(e)(5).

### FIELD OF THE INVENTION

The field of the present invention generally relates to the production and use of vaccines against pathogens.

### BACKGROUND OF THE INVENTION

Immunotherapeutic compositions, including vaccines, are one of the most cost-effective measures available to the health care industry for the prevention and treatment of disease. There remains, however, an urgent need to develop safe and effective immunotherapy strategies and adjuvants for a variety of diseases, including those caused by or associated with infection by pathogens. For the treatment of infectious diseases, including viral diseases and diseases caused by intracellular pathogens, it is desirable to provide immunotherapy that elicits a cell-mediated (cellular) immune response, although many vaccines are directed primarily or entirely to elicitation of humoral immunity. Indeed, a disadvantage of many subunit vaccines, as well as many killed or attenuated pathogen vaccines, is that while they appear to stimulate a strong humoral immune response, they fail to elicit protective cell-mediated immunity.

Pathogen specific CD8 T cells can provide protective responses in both animal models and in humans by, for example, killing infected cells (Harty, J. T., et al. 2000. Annu Rev Immunol 18:275-308). However, most current vaccines act by generating specific antibodies that either neutralize or otherwise inactivate the pathogen (Plotkin, S. A. 2008. Clin Infect Dis 47:401-409). The influenza vaccine acts in this way: antibodies to the surface protein, haemagglutinin (HA), bind and inactivate the virus before it can infect host cells, thereby providing sterilizing immunity; the most optimal form of immune protection. However, HA and the other major antibody target, neuramindase (NA), alter year by year as a result of mutation and reassortment of viral genes leading to viral escape from immunodetection (Webster, R. G., et al. 1992. Microbiol Rev 56:152-179). Consequently, a new vaccine containing the HA and NA proteins from the virus sub-type expected to be circulating in the forthcoming influenza season, must be developed annually. However, it not always possible to make accurate predictions in time to stock-pile enough influenza vaccine. Therefore, a vaccine that induced a more universally protective anti-influenza immune response is highly desirable.

CD8 T cell influenza epitopes are more likely to be found in the less variable internal core proteins of the virus and influenza specific cells have been found to recognize different viral sub-types (Boon, A. C., et al. 2004. J Immunol 172:2453-2460; Braciale, T. J. 1977. J Exp Med 146:673-689; Jameson, J., et al. 1999. J Immunol 162:7578-7583, Kees, U., and Krammer, P. H. 1984. J Exp Med 159:365-377; Townsend, A. R., et al. 1984. Cell 39:13-25; Yewdell, J. W., et al. 1985. Proc Natl Acad Sci USA 82:1785-1789). For this reason, influenza specific T cells can provide cross-reactive protection in mice and their presence is associated with reduced viral levels and disease in humans (Christensen, J. P., et al. 2000. J Virol 74:11690-11696; Epstein, S. L. 2006. J Infect Dis 193:49-53; Furuya, Y., et al. 2010. J Virol 84:4212-21; McMichael, A. J., et al. 1983. N Engl J Med 309:13-17; O'Neill, E., et al. 2000. J Gen Virol 81:2689-2696; Ulmer, J. B., et al. 1993. Science 259:1745-1749). Unlike antibody, CD8 T cells can only act to reduce disease following an infection as their activation requires processing and presentation of antigen on MHC class I molecules. Despite this, there has been growing interest in the development of vaccines that generate protective CD8 T cells as many diseases cannot be effectively controlled by antibody alone (Appay, V., et al. 2008. Nat Med 14:623-628; Doherty, P. C., and Kelso, A. 2008. J Clin Invest 118:3273-3275).

Vaccines fall into two main categories: those that contain the whole microorganism, which has either been attenuated or inactivated, and those that are made up of parts of the pathogen, these are known as sub-unit vaccines. While vaccines in the former category contain both antigen and innate-stimulating components that provide all the signals required to activate fully the adaptive immune response, sub-unit vaccines must be given with an adjuvant to provide an effective stimulatory environment (McKee, A. S. et al. 2010. BMC Biol 8:37).

Antigen specific CD8 T cells are activated following immunization of mice with protein and the aluminum salt, alyhdrogel (also referred to as alum) (McKee, A. S., et al. 2009. J Immunol 183:4403-4414). Alum causes destabilizaton of endocytic vesicles allowing co-injected antigens to enter the cytosol (Hornung, V., et al. 2008. Nat Immunol 9:847-856). This should allow all APC that phagocytose antigen delivered with alum to have the potential to present to CD8 T cells. That this is not the case suggests that simply the presence of antigen in the cytosol is not sufficient for the activation of CD8 T cells.

While neutralizing antibody offers the most optimal form of immunity to pathogens, immunization with a vaccine that generates protective CD8 T cells in addition to the current vaccine will create a double-layered shield of protection. The memory CD8 T cells will be able to clear rapidly any cells infected by virus that has escaped antibody-mediated control and thereby stop the infection before it can spread to other cells. These findings present a solution to the current problems of vaccines against pathogens.

### SUMMARY

Various embodiments of the invention are described below. However, the invention is not limited to embodiments described in this summary, as inventions described in the description that follows are also expressly encompassed.

The present invention provides for a composition comprising an isolated internal pathogenic protein, a Toll-like receptor (TLR) agonist and an aluminum salt. The isolated internal pathogenic protein can be selected from the group consisting of influenza nucleoprotein (NP), matrix protein 1 (M1), matrix protein 2 (M2), non-structural-1 (NS 1), non-structural-2 (NS2), polymerase acidic (PA), polymerase basic 1 (PB1), polymerase basic 1 F2 (PB2-F2), and polymerase basic 2 (PB2), and in a preferred embodiment, the isolated internal pathogenic protein is NP and further, the NP can be from influenza A.

Another aspect of the composition of the present invention provides that the isolated internal pathogenic protein is from a pathogen that can be selected from the group consisting of a virus, parasite and bacteria.

Another aspect of the composition of the present invention further provides that the TLR agonist is selected from the group consisting of TLR1, TLR2, TLR3, TLR4, TLR5, TLR6, TLR7, TLR8, TLR9, TLR10, TLR11, TLR12, and TLR13. In a preferred embodiment of the present invention, the TLR agonist is selected from the group consisting of a lipopolysaccharide (LPS) derivative or mimetic, monophosphoryl lipid A (MPL) or RC529. In another aspect of the composition of the present invention, the LPS derivative is MPL.

In yet another aspect of the composition of the present invention the aluminum salt is selected from the group consisting of alum, potassium aluminum sulfate, aluminum phosphate, and aluminum hydroxide. In a preferred aspect of the composition of the present invention the aluminum salt is alum.

In another aspect of the composition of the present invention, the composition may be administered to a subject orally, subcutaneously, intramuscularly, intravenously, by aerosol to the respiratory tract, or intradermally.

In a preferred aspect, the composition of the present invention comprising an influenza A nucleoprotein, MPL and alum.

The present invention also provides for a method for protecting a subject against infection by a pathogen comprising administering to the subject a composition comprising an isolated internal pathogenic protein, a Toll-like receptor (TLR) agonist and an aluminum salt. This method further provides that the pathogen can be capable of causing a disease selected from the group consisting of influenza, a rhinovirus associated disease, adenovirus associated disease, malaria and Listeria infection. This method further provides that the influenza can be selected from the group consisting of influenza A, influenza B and influenza C.

In another aspect, this method provides that the isolated internal pathogenic protein is selected from the group consisting of influenza nucleoprotein (NP), M1, M2, NS1, NS2, PA, PB1, PB1-F2, PB2. The method further provides that the isolated internal pathogenic protein can be NP. In a preferred embodiment of this method the NP is from influenza A. This method further provides that the TLR agonist is selected from the group consisting of TLR1, TLR2, TLR3, TLR4, TLR5, TLR6, TLR7, TLR8, TLR9, TLR10, TLR11, TLR12, and TLR13. This method further provides that the TLR agonist is selected from the group consisting of a lipopolysaccharide (LPS) derivative or mimetic, MPL or RC529. In a preferred embodiment the method provides that the LPS derivative is MPL.

In yet another aspect of the method of the present invention the aluminum salt is selected from the group consisting of alum, potassium aluminum sulfate, aluminum phosphate, and aluminum hydroxide. In a preferred aspect of the method the aluminum salt is alum.

In still another aspect of the method of the present invention provides that the route of administration may be intra-peritoneal (i.p.), oral, subcutaneous, intramuscular, intravenous, by aerosol to the respiratory tract, or intradermal.

The present invention also provides a method for protecting a subject against infection by influenza comprising administering to the subject a composition comprising an influenza A nucleoprotein, MPL and alum.

In yet another aspect of the methods of the present invention the subject is human.

### BRIEF DESCRIPTION OF THE DRAWINGS

FIGS. 1A-1C show that protein delivered with alum primes antigen specific memory CD8 T cells. In FIG. 1A B6 mice were immunized with OVA+ alum or were not immunized and the percent of Kb/SIINFEKL (SEQ ID NO:1) tetramer+ cells in the spleen was examined 9 days later. Cells are gated on live CD8+ lymphocytes that were negative for B220, CD4, F4/80 and MHC class II. The numbers are the percent of cells within the indicated gate. The data shown are representative of 8 experiments with 3-4 mice/group. In FIG. 1B the experiments were performed as in FIG. 1A, but the phenotype of the Kb/SIINFEKL (SEQ ID NO:1) tetramer+ cells 60 days after immunization was examined by flow cytometry. Cells were gated on CD44hi Kb/SIINFEKL (SEQ ID NO:1) tetramer+ gates (light grey line) or CD8+ cells (closed grey histogram with dark line). The data are representative of 3-4 experiments with 3-4 mice/group. In FIG. 1C the experiments were performed as in FIG. 1A, but the numbers of Kb/SIINFEKL (SEQ ID NO:1) tetramer+ cells were examined at various times after immunization, cells were gated as in FIG. 1A. The horizontal line indicates the level of detection as determined by staining spleen cells from naïve age-matched mice. The data are from two experiments with a total of 8 mice per time point.

FIGS. 2A-2D show that CD8, but not CD4, T cell responses are reduced in the absence of Batf3. In FIG. 2A Batf3 heterozygous (HET) and deficient (KO) mice were primed with 3K-OVA+ alum intraperitoneal (i.p.) and the percent of CD4 T cells that bound to IAb/3K tetramer examined 8 days later. Cells were gated on live CD4+ lymphocytes negative for B220, CD8, F4/80 and MHC class II. The number shows the percent of IAb/3K tetramer+CD44hi cells in the adjacent gates. In FIG. 2B the number of IAb/3K tetramer+CD44hi cells in the mice in FIG. 2A and similarly treated wild-type B6 (WT) mice was calculated. Each symbol represents a mouse and the line shows the mean of the group. The data are representative of 3 experiments with 3 mice per group. In FIG. 2C the percent of CD8 T cells that bound to the Kb/SIINFEKL (SEQ ID NO:1) tetramer was examined on mice treated as in FIG. 2A. The number shows the percent of Kb/SIINFEKL (SEQ ID NO:1)+CD44hi cells in the adjacent box. Cells are gated on live CD8+ lymphocytes negative for B220, CD4, F4/80 and MHC class II. In FIG. 2D the number of Kb/SIINFEKL (SEQ ID NO:1)+CD44hi cells in the mice in FIG. 2A and similarly treated wild-type B6 (WT) mice was calculated. Each symbol represents a mouse and the line shows the mean of the group. The data are representative of 3 experiments with 3 mice per group.

FIGS. 3A and 3B show that alum activated CD8 T cells make IFNγ not IL4. In FIG. 3A 4Get mice on the B6 background, were immunized with 3K-OVA+ alum i.p. Eight days later the percents of IAb/3K tetramer+(out of CD4 cells) or Kb/SIINFEKL (SEQ ID NO:1) tetramer+ cells (out of CD8 cells) in the spleen were measured and the percentages of these cells that were GFP+ determined (indicated as “primary” on the figure). Alternatively, the mice were rested for 100 days then re-immunized with 3K-OVA+ alum. 5 days later, the percents of IAb/3K tetramer or Kb/SIINFEKL (SEQ ID NO:1) tetramer+ cells in the spleen were measured and the percent of these cells that were GFP+ cells determined (indicated as “recall” on the figure). Cells were gated either on CD4+ live lymphocytes that were B220, CD8, F4/80 and MHC II negative (row 1) and on the IAb/3K tetramer CD44hi cells (row 2). In row 3 the cells were gated as in FIG. 1A and on Kb/SIINFEKL (SEQ ID NO:1) tetramer+CD44hi cells in row 4. Numbers shown are the percent of cells within the indicated gate or quadrant. The data shown are representative of 1-2 experiments with 4 mice/group. In FIG. 3B eight days after B6 mice were primed with OVA+ alum their splenocytes were activated with SIINFEKL (SEQ ID NO:1) peptide for 6 hours in the presence of Golgi plug or with Golgi plug alone. The cells in the plots are gated on CD8+ B220-CD4-MHC class II-cells. The number indicates the percent of cells in the indicated gate that were CD44hi IFNγ+. The data shown are representative of 8 experiments with 3-4 mice per group.

FIGS. 4A-4D show that mice primed with a CD8 epitope from influenza nucleoprotein, alum and MPL are protected from influenza A infection. In FIG. 4A B6 mice were immunized with BSA/NP+ alum or were not immunized and the percent of Db/NP tetramer+ cells in the spleen was examined 9 days later. Cells were gated as in FIG. 1A. The numbers indicate the percent of cells in the adjacent gate. Plots are representative of 2 experiments with 3-4 mice/group. In FIG. 4B B6 mice were immunized with NP/OVA or NP/BSA either alone (closed diamonds) or with alum (open diamonds), MPL (open squares) or both alum and MPL (closed squares) or with the two adjuvants and PBS (closed triangles). 5-14 weeks later, the mice were infected with 150 plaque forming units (PFU) of influenza A intranasally (i.n.) and weighed every day. The data shown are combined from 2 experiments with 4-5 mice/group. In FIG. 4C the experiments were performed as in FIG. 4B, but the numbers of Db/NP tetramer+ cells, in one lobe of the lung, were examined on day 4 by flow cytometry. The data shown were combined from 3 experiments with 4-5 mice/group. Cells were gated as in FIG. 1A and error bars show SEM. In FIG. 4D the experiments were performed as in FIG. 4A, but the viral titers in one lobe of the lung were measured on day 4 as described in the Examples section. The data were combined from 3 experiments, error bars show SEM.

FIG. 5 shows that protected and control mice have similar numbers of antigen specific cells in the MLN and lung 8 days after infection. The experiments were performed as in FIG. 4 but MLN (A and C) or lungs (B and D) were taken 8 days after infection with influenza A and the cells stained with Db/NP tetramer (A and B) or Db/PA tetramer (C and D) to count the number of antigen specific cells. Each symbol represents a mouse and the line shows the mean of the group. The horizontal line indicates the level of detection found by staining cells from naïve mice with the class I tetramer. The data are representative of 1 of 2 experiments with 4/5 mice/group.

FIGS. 6A-6E show the addition of MPL does not affect T cell priming but enhances cytotoxic differentiation. In FIG. 6A B6 mice were immunized with OVA+ alum, MPL or both adjuvants and the numbers of Kb/SIINFEKL (SEQ ID NO:1) tetramer+ cells in spleens examined. Cells were gated as in FIG. 1A. The horizontal line indicates the level of detection. The data shown are representative of 2 experiments with 4 mice/group. In FIG. 6B B6 mice were primed with NP/OVA or NP/BSA with the indicated adjuvant and the numbers of Db/NP tetramer+ cells in spleens examined 6-14 weeks later. Each symbol represents a mouse and the line shows the group mean. The horizontal line indicates the level of detection. The data are from 3 experiments with 3-4 mice/group. In FIG. 6C B6 mice were primed with OVA and alum, MPL or both adjuvants i.p. 7 days later these mice and naïve mice received a 1:1 mixture of splenocytes half of which had been incubated with SIINFEKL (SEQ ID NO:1) peptide and stained with a high level of CFSE and the second half stained with a low level of CFSE. Two days later, the percent of total CFSE positive cells that were CFSEhi was examined in the spleen. A representative sample from each group is shown. The number indicates the percent of CFSEhi cells out of total CFSE+ cells. In FIG. 6D B6 and IL6 knockout mice were immunized with OVA delivered with alum or alum+ MPL i.p. and 7 days later these mice received SIINFEKL (SEQ ID NO:1)-loaded, CFSE stained cells as in FIG. 6C and the percent of CFSEhi peptide loaded cells out of total CFSE+ cells in the spleen examined 2 days later. Each symbol represents one mouse and the line shows the mean of the group, n.s.: not significant. In FIG. 6E B6 and perforin knockout mice were primed with NP/OVA with alum+ MPL or B6 mice were given adjuvant alone. 6 weeks later, these mice were infected with 150 PFU of influenza A i.n and the mice weighed daily. The data are representative of 1 of 2 experiments with 5 mice/group.

FIGS. 7A-7E shows PD 1 upregulation on alum primed cells inhibits cytotoxic differentiation. In FIG. 7A B6 mice were primed with OVA and alum, MPL or both adjuvants i.p. and the levels of PD1 expression on the surface of Kb/SIINFEKL (SEQ ID NO:1) tetramer positive cells examined 8 days later. Cells are gated on Kb/SIINFEKL (SEQ ID NO:1)+CD44hi cells (open histograms) or on total CD8 T cells from a naïve animal (filled histogram). In FIG. 7B B6 mice were treated as in FIG. 7A but the MFI of PD1 on Kb/SIINFEKL tetramer positive cells was examined at various time points after immunization. *=p<0.05, **=p<0.01, ***=p<0.001. In FIG. 7C B6 mice were primed with OVA and alum i.p. on day 0 and treated with anti-PDL1 or an isotype control antibody on days 0, 3 and 7. The number of Kb/SIINFEKL (SEQ ID NO:1) tetramer positive cells present in the spleen was examined 8 days later. In FIG. 7D B6 mice were treated as in FIG. 7C or left naive and received CFSE labeled cells half of which had been pulsed with SIINFEKL (SEQ ID NO:1) peptide as in FIG. 6C on day 7. 48 hours later, the percent of CFSEhi/SIINFEKL (SEQ ID NO:1) pulsed cells out of total CFSE+ cells was examined. In FIG. 7E B6 mice were primed with OVA and alum, MPL or both adjuvants i.p. on day 0 and some of the mice treated with an isotype control antibody or anti-PD1 on days 0, 3 and 7. The percent of Kb/SIINFEKL (SEQ ID NO:1) tetramer positive cells expressing granzyme B was examined 8 days later.

FIG. 8 shows that MPL does not alter the number of IFNγ produced after immunization with OVA and alum. Eight days after B6 mice were primed with OVA with alum, MPL or both adjuvants, their splenocytes were incubated ex vivo with SIINFEKL (SEQ ID NO:1) in the presence of Golgi Plug for 6 hours or with Golgi plug alone. The numbers of CD8+CD4−MHCII−B220− that were IFNγ+ were examined by intracellular staining. Each symbol represents one mouse and the line shows the mean of the group. The horizontal line indicates the level of background staining of spleen cells cultured with Golgi Plug in the absence of peptide. The data are representative of 3 experiments with 4 mice per group in each experiment.

FIG. 9 shows that MPL enhances the cytotoxic response of alum-primed CD8 T cells. B6 mice were primed with OVA and alum, MPL or both adjuvants i.p. 7 days later these mice and naïve mice received a 1:1 mixture of splenocytes half of which had been incubated with SIINFEKL (SEQ ID NO:1) peptide and stained with a high level of CFSE and the second half stained with a low level of CFSE. Two days later, the percent of total CFSE positive cells that were CFSEhi was examined in the spleen. Each symbol represents one mouse and the line shows the mean of the group. The data are combined from 2 experiments.

FIG. 10 shows that anti-PDL1 treatment does not affect the number of IFNγ producing cells activated after immunization with alum and antigen B6 mice were primed with OVA and alum i.p. on day 0 and treated with anti-PDL1 or an isotype control antibody on days 0, 3 and 7. Eight days later, spleen cells were activated with SIINFEKL (SEQ ID NO:1) peptide in the presence of Golgi plug and the number of IFNγ+CD8+ cells calculated. Each symbol represents a mouse and the line shows the mean of the group. Data is representative of two experiments with 4 mice per group. The horizontal line indicates the level of background staining of spleen cells incubated with Golgi plug in the absence of peptide.

### DESCRIPTION OF INVENTION

The present invention generally relates to compositions and methods for protection against infection by a pathogen. The invention includes a composition comprising an isolated internal pathogenic protein, a toll-like receptor agonist and an aluminum salt, as well as the use of such compositions for protecting a subject against infection by a pathogen.

CD8 T cells can provide protective responses in both animal models and in humans. CD8 T cell epitopes are much less variable and thus a vaccine designed to activate protective CD8 T cells has the potential to protect against yearly and newly emerging pandemic viral subtypes. For example, the CD8 T cells in both mouse and man cross-react across different sub-types of influenza virus. Therefore, a vaccine that generates CD8 T cells may provide cross-reactive protection in influenza. Furthermore, one of the major risk groups for influenza infection are the elderly who make poor B cell responses to new vaccines. By generating CD8 memory T cells, these individuals may be more likely to be protected. The presence of granzyme expressing influenza specific CD8 T cells provides a better correlate of protection from influenza in elderly individuals than the presence of HA specific antibodies.

For example, the internal pathogenic protein influenza nucleoprotein (NP) is highly conserved between different strains of the influenza A viruses and human CD8 T cells reactive to a number of epitopes within nucleoprotein respond to a range of viruses including H5N1 virus subtypes. The cross reactivity of NP specific CD8 T cells for different strains of influenza A has been recognized and such cells have been shown to provide protection from heterosubtypic influenza challenge by a number of groups. Therefore, immunization with an isolated internal pathogenic protein such as NP with an aluminum salt such as alum and a TLR agonist such as MPL will generate cross-reactive cytotoxic lymphocytes that can provide protection in the majority of individuals to most, if not all, influenza A subtypes. Importantly, such T cells are thought to protect against the worst forms of disease that occur in those that have no cross-reactive antibody response. Therefore, a vaccine composition of the present invention has clear advantages over other current vaccines because many of these vaccines induce protective antibodies to viral surface proteins but do not offer protections against annual variants or emerging pathogenic strains.

The present invention demonstrates that a composition comprising an isolated internal pathogenic protein, and aluminum salt and a TLR agonist results in a protective response to infection in vivo. Without being bound by theory, the generation of memory CD8 T cells is activated with antigen delivered with aluminum salt. The resultant memory cells are long-lived and can protect animals from disease following infection with the pathogen from which the pathogenic protein is derived. Without being bound by theory, the addition of MPL enhances protection by increasing the differentiation of the activated T cells into cytotoxic cells (CTLs), a process that requires interleukin (IL) 6 and results in the down-regulation of PD1 (programmed death 1 receptor) on the surface of the T cells primed in the presence of alum. The increased PD1 expression on T cell primed in the presence of alum reduced the cytotoxic response as blocking interactions between PD1 and PDL1 (programmed death ligand) increase granzyme B expression and improve the cytotoxic response of the CD8 T cells.

Before the present invention is further described, it is to be understood that this invention is not limited to particular embodiments described, as such may, of course, vary it is also to be understood that the terminology used herein is for the purpose of describing particular embodiments only, and is not intended to be limiting, since the scope of the present invention will be limited only by the claims. It must be noted that as used herein and in the claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a host factor refers to one or more host factors. As such, the terms “a”, “an”, “one or more” and “at least one” can be used interchangeably. Similarly the terms “comprising”, “including” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

One embodiment of the present invention is a composition comprising an isolated internal pathogenic protein, a TLR agonist and an aluminum salt. As used herein the term “internal pathogenic protein” refers to any non-surface proteins of a pathogen and includes whole proteins, internal portions of transmembrane proteins, peptides, and protein fragments. Compositions of the present invention in some embodiments can include two or more isolated internal pathogenic proteins.

In various embodiments, the isolated internal pathogenic protein encompassed by the invention can include at least a portion or the full-length of any one or more influenza proteins selected from nucleoprotein (NP), matrix protein 1 (M1), matrix protein 2 (M2), non-structural-1 (NS1), non-structrual-2 (NS2), polymerase acidic (PA), polymerase basic 1 (PB1), polymerase basic 1 F2 (PB2-F2), and polymerase basic 2 (PB2) and/or any one or more immunogenic domains of any one or more of these influenza proteins. In a preferred embodiment, the isolated internal pathogenic protein is the influenza A NP protein.

Isolated internal pathogenic proteins of the present invention can be derived from any suitable pathogen. The pathogen can be a virus, parasite or bacteria. In some embodiments, the internal pathogenic protein is derived from rhinovirus. In some embodiments the isolated internal pathogenic protein is derived from adenovirus. In some embodiments the isolated internal pathogenic protein is derived from a parasite (such as the parasite that causes malaria). In some embodiments the isolated internal pathogenic protein is derived from a bacteria (such as Listeria).

In the compositions of the present invention the isolated internal pathogenic protein can function as an antigen. According to the present invention, the general use herein of the term “antigen” refers: to any portion of a protein (peptide, partial protein, full-length protein), wherein the protein is naturally occurring or synthetically derived, to a cellular composition (whole cell, cell lysate or disrupted cells), to an organism (whole organism, lysate or disrupted cells) or to a carbohydrate, or other molecule, or a portion thereof. An antigen may elicit an antigen-specific immune response (e.g., a humoral and/or a cell-mediated immune response) against the same or similar antigens that are encountered by an element of the immune system (e.g., T cells, antibodies).

An antigen can be as small as a single epitope, or larger, and can include multiple epitopes. As such, the size of an antigen can be as small as about 5-12 amino acids (e.g., a peptide) and as large as: a full length protein, including a multimer and fusion proteins, chimeric proteins, whole cells, whole microorganisms, or portions thereof (e.g., lysates of whole cells or extracts of microorganisms). In addition, antigens can include carbohydrates, which can be loaded into a composition of the invention. It will be appreciated that in some embodiments, the antigen is a protein, fusion protein, chimeric protein, or fragment thereof, rather than an entire cell or microorganism.

An “immunogenic domain” of a given antigen can be any portion, fragment or epitope of an antigen (e.g., a peptide fragment or subunit or an antibody epitope or other conformational epitope) that contains at least one epitope that acts as an immunogen when administered to an animal. For example, a single protein can contain multiple different immunogenic domains. Immunogenic domains need not be linear sequences within a protein, such as in the case of a humoral immune response.

An epitope is defined herein as a single immunogenic site within a given antigen that is sufficient to elicit an immune response. Those of skill in the art will recognize that T cell epitopes are different in size and composition from B cell epitopes, and that epitopes presented through the Class I MHC pathway differ from epitopes presented through the Class II MHC pathway. Epitopes can be linear sequence or conformational epitopes (conserved binding regions).

The invention also includes homologues of any of the above-described internal pathogenic proteins, as well as the use of homologues, variants, or mutants of the individual internal pathogenic proteins or portions thereof.

In various embodiments of the present invention, the TLR agonist may be a TLR1, TLR2, TLR3, TLR4, TLR5, TLR6, TLR7, TLR8, TLR9, TLR10, TLR11, TLR12, or TLR13 agonist. Examples of such an agonist include, without limitation, a lipopolysaccharide (LPS) derivative or mimetic, MPL (monophosphoryl lipid A) or RC529.

In various embodiments of the present invention the aluminum salt is alum, potassium aluminum sulfate, aluminum phosphate, or aluminum hydroxide. In a preferred embodiment the aluminum salt is alum.

While various examples of the present invention are described in detail below, it is apparent that modifications and adaptations of those examples will occur to those skilled in the art. It is to be expressly understood, however, that such modifications and adaptations are within the scope of the present invention. The inventors have demonstrated that a composition comprising an isolated internal pathogenic protein adjuvanted with alum, protects mice from some of the weight loss associated with influenza infection. Addition of the TLR agonist, MPL to the formulation did not alter the number of CD8 memory cells generated with alum alone but improved protection by increasing the differentiation of cytotoxic cells. This protection was observed by a reduction in both weight loss and viral titers following infection. While antigen (isolated internal pathogenic protein) and MPL alone activated a good primary response including substantial cytotoxic T cell differentiation, these cells were unable to protect mice from subsequent influenza infection. This was due to the poor survival of the memory cells generated by this immunization. Therefore, alum and MPL complement each other: alum provides signals required for the generation of long-lived memory cells while MPL enhances cytotoxic differentiation.

As demonstrated in the Example section the inventors have shown that immunization with an isolated internal pathogenic protein (antigen) and alum primes CD8 T cells that differentiate into IFNγ producing cells but have only limited cytotoxic potential. The signals that induce these two CD8 T cell effector functions are not explicably linked. Co-injection of MPL increased granzyme B expression by the alum-primed cells and the increased cytotoxic response required IL6. Currently, it is not clear how the IL6 acts and on which cell type. MPL does enhance the production of IL6 at the injection site when delivered with alum. This implies that the IL6 would act on presenting cells that migrate from this site to secondary lymphoid organs. However, such cells do not differentiate into the CD8α+DC required to prime CD8 T cells. Alternatively, IL6 may be produced in the spleen and act either on the priming APC or directly on the activated CD8 T cells. In support of this, IL6 can enhance the differentiation of cytotoxic CD8 T cells in the presence of IL2 in vitro following activation of purified CD8 T cells with anti-CD3 and anti-CD28 (32).

Importantly, the inventors have shown that MPL also reduces the expression of PD1 on the alum-primed CD8 T cells and that this contributes to its ability to enhance the CD8 T cell cytotoxic response. The high expression of PD1 by the alum-primed CD8 T cells led to a reduction in granzyme B expression, and therefore the majority of these cells did not differentiate into cytotoxic cells. Consequently, when the memory cells generated from this response were re-activated by the influenza A infection, they first had to differentiate into cytotoxic cells before they could provide protection, potentially explaining why these cells provided less protection than those primed with both alum and MPL.

As CD8 T cells can only recognize a virus following active infection of host cells, the immunization did not prevent infection following challenge. As a result of this, a primary immune response to other epitopes of the virus occurred. For example, the numbers of CD8 T cells responding to the PA₂₂₄₋₂₃₃ epitope were similar in immunized and control animals. Infection in the absence of severe disease may actually provide long-term advantages. A low level of infection, as seen here in mice immunized with NP/protein and both an aluminum salt and TLR, will allow the immune system to be primed or boosted to the virus, enhancing future protection whilst preventing disease.

In one embodiment of the present invention, a composition can include additional agents, which may also be referred to as biological response modifier compounds, or the ability to produce such agents/modifiers. Biological response modifiers include additional adjuvants and other compounds that can modulate immune responses, which may be referred to as immunomodulatory compounds, as well as compounds that modify the biological activity of another compound or agent, such biological activity not being limited to immune system effects. Certain immunomodulatory compounds can stimulate a protective immune response whereas others can suppress a harmful immune response, and whether an immunomodulatory is useful in combination with the composition may depend, at least in part, on the disease state or condition to be prevented, and/or on the individual who is to be treated. Certain biological response modifiers preferentially enhance a cell-mediated immune response whereas others preferentially enhance a humoral immune response (i.e., can stimulate an immune response in which there is an increased level of cell-mediated compared to humoral immunity, or vice versa.). There are a number of techniques known to those skilled in the art to measure stimulation or suppression of immune responses, as well as to differentiate cell-mediated immune responses from humoral immune responses, and to differentiate one type of cell-mediated response from another (e.g., a TH17 response versus a TH1 response).

Agents/biological response modifiers useful in the invention may include, but are not limited to, cytokines, chemokines, hormones, lipidic derivatives, peptides, proteins, polysaccharides, small molecule drugs, antibodies and antigen binding fragments thereof (including, but not limited to, anti-cytokine antibodies, anti-cytokine receptor antibodies, anti-chemokine antibodies), vitamins, polynucleotides, nucleic acid binding moieties, aptamers, and growth modulators.

Agents can include agonists and antagonists of a given protein or peptide or domain thereof. As used herein, an “agonist” is any compound or agent, including without limitation small molecules, proteins, peptides, antibodies, nucleic acid binding agents, etc., that binds to a receptor or ligand and produces or triggers a response, which may include agents that mimic the action of a naturally occurring substance that binds to the receptor or ligand. An “antagonist” is any compound or agent, including without limitation small molecules, proteins, peptides, antibodies, nucleic acid binding agents, etc., that blocks or inhibits or reduces the action of an agonist.

Accordingly, in one embodiment the present invention includes a method for protecting a subject against infection by a pathogen comprising administering to the subject a composition comprising an isolated internal pathogenic protein, a TLR and an aluminum salt. In another embodiment, the present invention the pathogen is capable of causing a disease including but not limited to, influenza (influenza A, B and C), rhinovirusal related diseases (the common cold), adenovirus related diseases (respiratory diseases such as common cold, pneumonia, croup, and bronchitis as well as infection of the stomach and intestine (gastroenteritis), eyes (conjunctivitis), and bladder (cystitis)) and malaria.

For a subject who has a viral infection, viral disease (e.g., a viral-associated disease) or parasitic disease, it is generally desirable to achieve the benefits of a TH1 and CD8+ immune response, particularly in individuals who are resistant to interferon-driven therapy, and so with this type of infection or disease (which is expected to be applicable to other intracellular pathogens), the composition may be administered to induce a CD8+ immune response in the subject.

Another embodiment of the invention relates to a method to immunize an individual or population of individuals against a pathogen in order to prevent pathogenic infection, and/or reduce the severity of pathogenic infection in the individual or population of individuals. The method includes the step of administering to an individual or population of individuals that is not infected with the pathogen (or believed not to be infected with the pathogen), a composition of the invention.

The composition of one embodiment of the invention may be administered using techniques well known to those in the art. Preferably, compounds are formulated and administered by genetic immunization. Techniques for formulation and administration may be found in “Remington's Pharmaceutical Sciences”, 18^{th} ed., 1990, Mack Publishing Co., Easton, Pa. Suitable routes may include parenteral delivery, such as intramuscular, intradermal, subcutaneous, intramedullary injections, as well as, intrathecal, direct intraventricular, intravenous, intraperitoneal, intranasal, or intraocular injections, just to name a few. Other routes include oral or transdermal delivery. For injection, the composition of one embodiment of the invention may be formulated in aqueous solutions, preferably in physiologically compatible buffers such as Hanks' solution, Ringer's solution, or physiological saline buffer.

For parenteral application, which includes intramuscular, intradermal, subcutaneous, intranasal, intracapsular, intraspinal, intrasternal, and intravenous injection, particularly suitable are injectable, sterile solutions, preferably oily or aqueous solutions, as well as suspensions, emulsions, or implants, including suppositories. Formulations fix injection may be presented in unit dosage form, e.g., in ampoules or in multi-dose containers, with an added preservative. The compositions may take such forms as suspensions, solutions or emulsions in oily or aqueous vehicles, and may contain formulator agents such as suspending, stabilizing and/or dispersing agents. Alternatively, the active ingredient may be in powder form for constitution with a suitable vehicle, e.g., sterile pyrogen-free water, before use.

For enteral application, particularly suitable are tablets, dragees, liquids, drops, suppositories, or capsules. The pharmaceutical compositions may be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. A syrup, elixir, or the like can be used wherein a sweetened vehicle is employed.

Sustained or directed release compositions can be formulated, e.g., liposomes or those wherein the active compound is protected with differentially degradable coatings, e.g., by microencapsulation, multiple coatings, etc. It is also possible to freeze dry the new compounds and use the lyophilizates obtained, for example, for the preparation of products for injection.

For administration by inhalation, the compounds for use according to one embodiment of the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch. For topical, or transdermal, application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient; preferably in combination with a solid or liquid inert carrier material, is packaged in a squeeze bottle or in admixture with a pressurized volatile, normally gaseous propellant, e.g., a freon. The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

In accordance with one embodiment of the present invention the compositions may comprise a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g., intravenous, cutaneous or subcutaneous, intramucosal (e.g., gut), intranasal, intramuscular, or intraperitoneal routes.

“Subject” refers to any member without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention is intended for use involving any of the above vertebrate species, since the immune systems of all of these vertebrates operate similarly.

In general, the term “biologically active” indicates that a compound (including a protein or peptide) has at least one detectable activity that has an effect on the metabolic or other processes of a cell or organism, as measured or observed in vivo (i.e., in a natural physiological environment) or in vitro (i.e., under laboratory conditions).

According to the present invention, the term “modulate” can be used interchangeably with “regulate” and refers generally to upregulation or downregulation of a particular activity. As used herein, the term “upregulate” can be used generally to describe any of: elicitation, initiation, increasing, augmenting, boosting, improving, enhancing, amplifying, promoting, or providing, with respect to a particular activity. Similarly, the term “downregulate” can be used generally to describe any of: decreasing, reducing, inhibiting, ameliorating, diminishing, lessening, blocking, or preventing, with respect to a particular activity.

Reference to a protein or polypeptide used in the present invention includes full-length proteins, fusion proteins, or any fragment, domain, conformational epitope, or homologue of such proteins. More specifically, an isolated protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein of the present invention is produced recombinantly. According to the present invention, the terms “modification” and “mutation” can be used interchangeably, particularly with regard to the modifications/mutations to the amino acid sequence of proteins or portions thereof (or nucleic acid sequences) described herein.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein. Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

A homologue of a given protein may comprise, consist essentially of, or consist of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to the amino acid sequence of the reference protein. In one embodiment, the homologue comprises, consists essentially of, or consists of, an amino acid sequence that is less than 100% identical, less than about 99% identical, less than about 98% identical, less than about 97% identical, less than about 96% identical, less than about 95% identical, and so on, in increments of 1%, to less than about 70% identical to the naturally occurring amino acid sequence of the reference protein.

An isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes that are naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

A recombinant nucleic acid molecule is a molecule that can include at least one of any nucleic acid sequence encoding any one or more proteins described herein operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which is administered to an animal.

A recombinant nucleic acid molecule includes a recombinant vector, which is any nucleic acid sequence, typically a heterologous sequence, which is operatively linked to the isolated nucleic acid molecule encoding a fusion protein of the present invention, which is capable of enabling recombinant production of the fusion protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a virus or a plasmid. Recombinant vectors can be used in the cloning, sequencing, and/or otherwise manipulating of nucleic acid molecules, and can be used in delivery of such molecules (e.g., as in a DNA composition or a viral vector-based composition). Recombinant vectors are preferably used in the expression of nucleic acid molecules, and can also be referred to as expression vectors. Preferred recombinant vectors are capable of being expressed in a transfected host cell.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more expression control sequences. The phrase “operatively linked” refers to linking a nucleic acid molecule to an expression control sequence in a manner such that the molecule is expressed when transfected (i.e., transformed, transduced or transfected) into a host cell.

Various methods of the invention protect a subject from a pathogenic disease by administering compositions of the invention. As used herein, the phrase “protect a subject”, or any permutation thereof can generally refer to preventing a disease, preventing at least one symptom of the disease, delaying onset of a disease, reducing one or more symptoms of the disease, reducing the occurrence of the disease, and/or reducing the severity of the disease. The methods of the invention can result in one or more of: prevention of the disease or condition, prevention of infection, delay of the onset of disease or symptoms caused by the infection, increased survival, reduction of pathogen burden (e.g., reduction of viral titer), reduction in at least one symptom resulting from the infection in the individual, reduction of organ or physiological system damage resulting from the infection or disease, improvement in organ or system function, and/or improved general health of the individual.

The foregoing description of the present invention has been presented for purposes of illustration. The description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed.

### Example 1

### Mice, Immunizations and Infections

All animals were handled in strict accordance with good animal practice as defined by the relevant national and/or local animal welfare bodies, and all animal work was approved by the National Jewish Health Animal Care and Use Committee. Female B6, perforin knockout and interleukin 6 knockout mice were obtained from the Jackson Laboratory. B6 IL4 reporter (4Get) mice were obtained from Dr R. Locksley (University of California, San Francisco, Calif.) and Batf3 knockout mice were obtained from Ken Murphy (Washington University, St Louis, Mo.). Both these strains were bred at National Jewish Health in a specific-pathogen-free environment. Mice were age-matched within experiments and primed at 6-10 weeks of age. Mice were immunized with 10 μg of OVA protein (Sigma) or OVA-peptide or BSA-peptide conjugates. OVA or BSA proteins were conjugated to either 3K peptide or NP peptide as described (McKee, A. S., et al. 2009. J Immunol 183:4403-4414) using Malemide activated OVA or BSA (Pierce). Peptides were supplied by JPT (Berlin, Germany). All immunization were given i.p. and mice were injected with 1 mg of alum (Alydrogel, Brenntag) with or without 10 μg of MPL (Invivogen). Proteins were tumbled with alum with/out MPL for 2 hours at room temperature prior to injection. Infections with PR8 were done on mice anesthetized with isofluorane and 50 μl of PBS containing 150 PFU of virus injected i.n. The virus was prepared as described Wang, J., et al. 2009. J Immunol 182:1296-1304).

### Flow Cytometry

To count the numbers of antigen-specific T cells that were generated by the vaccine protocols used, tetramers made up of the MHC proteins Kb, Db or IAb, bound to antigenic peptides (from ovalbumen, SIINFEKL (SEQ ID NO:1), or influenza nucleoprotein, NP, or influenza polymerase A protein, PA) and colored with fluorescent proteins (allophycocyanin, APC or phycoerythrin, PE). These tetramers bind only to T cells that are specific for the MHC protein/peptide combination and make the relevant T cell fluorescent. Fluorescently labeled antibodies to CCR7 were used to determine which T cells bore CCR7 on their surfaces.

Splenocytes were removed at the indicated time, a single cell suspension prepared and red blood cells lysed. APC-K^{b}/SIINFEKL (SEQ ID NO:1) tetramer, APC-D^{b}/NP, PE-D^{b}/PA and PE-IA^{b}/3K tetramer were produced as described (Crawford, F., et al. 1998. Immunity 8:675-682). Single cell suspensions were stained with MHC tetramers at 37° C. for 2 hours. Antibodies to surface markers were added and the cells incubated for a further 20 minutes at 4° C., except for anti-CCR7 that was also incubated at 37° C. for 2 hours. Anti-CD127-PE, anti-CD4 and anti-CD8 APC-Cy7 and streptavidin PeCy7 (BD Biosciences). Anti-B220-pacific blue, anti-F4/80-pacific blue, anti-CD44 PerCP-Cy5.5 and anti-CCR7 biotin were from eBioscience. Anti-CD44 (IM3) Alexa 488, anti-CD122 (TMI31.4) Alexa 488, anti-class MHC II (Y3P) Alexa 405, anti-IFN-γ (XMG1.2) Alexa 647 were produced in the laboratory at National Jewish Health using Alexa 488, 405 or 657 protein conjugation kits (Molecular Probes). Tetramer+ cells were defined by gating on live (based on forward-side scatter characteristics), CD8 or CD4+ cells that were B220, F4/80, MHC class II negative and either CD4 or CD8 negative respectively.

To examine CD8 T cells in the lung, euthanized mice were perfused to remove blood from the lungs and one lobe cut into small pieces and treated with collagenase and DNase prior to flow staining as described above.

For analysis of cell proliferation using BrdU, mice were given drinking water containing 0.8 mg/ml of BrdU for 2 days before sacrifice. BrdU+ drinking water was replaced daily and protected from light at all times. Staining was carried out as described (Lenz, D. C., et al. 2004. Proc Natl Acad Sci USA 101:9357-9362) after the cells had been stained with tetramer and surface antibodies. Anti-BrdU Alexa-488 and Alexa 647 were from Molecular Probes.

For granzyme B staining, cells were stained with class I tetramer and surface antibodies then fixed with 4% paraformaldehyde then stained with anti-human granzyme B (BD) for 40 minutes.

For analysis of cytokine production by intracellular staining, splenocytes were activated ex vivo with 1 μg/ml SIINFEKL (SEQ ID NO:1) and 1 μg/ml of Golgi plug (BD Biosciences) for 6 hours. Cells were stained with surface antibodies and then fixed and permeabilized using the BD Cytofix/Cytoperm kit according to manufacturer's instructions. Antigen specific cytokine was defined as staining above the level of staining from splenocytes cultured in the absence of peptide. Cells were gated on live CD8+ cells that were CD4, B220 and MHC class II negative. In all cases, 2-5 million events were collected on a CyAn ADP (Dakocytomation), and data analyzed using FlowJo version 8.8 (Treestar).

### In Vivo Antibody Treatment

To block PD1-PDL1 interactions in vivo, mice were given 200 μg of anti-PDL1 (10F.9G2 from BioXCell) on days 0, 3 and 7 i.p. Control antibody was anti-DR5 (HB-151, grown and purified at NJH) and delivered as for anti-PDL1.

### In Vivo Killing Assay

A single-cell suspension of B6 splenocytes was prepared and red cell lysed. Half the cells were labeled with 10 μg/ml SIINFEKL (SEQ ID NO:1) at 37° C. for 2 hours, then stained with 1 μM CFSE (Molecular Probes) for 7 minutes at room temperature. The rest of the cells were labeled with 0.05 μM CFSE. Cells were washed and a 1:1 mixture of cells at a total of 5×10⁶ cells injected i.v. into naïve or immunized mice. 48 hours later, the percent of CFSE^{hi} out of total CFSE+ cells was measured by flow cytometery.

### Plaque Assay

One lobe of the lung from mice infected 4 days previously was homogenized and supernatants frozen until use. 1 μg/ml TPCK trypsin was added to the diluted supernatant which was plated on confluent MDCK cells. The cells were incubated for 1 hour at 37° C., the supernatants removed, then 1% seakem agar containing media added and plates returned to 37° C. 48 hours later, agar containing neutral red was added and plaques counted after a further 24-36 hours of incubation at 37° C.

### Statistics

To test for a significant drop in weight loss following infection, the percent weight loss was calculated for each mouse on each day following infection. The data was graphed and the area under the curve calculated for each mouse. Statistical significance determined using Student's two-tailed T test with GraphPad Prism software version 4.

### Example 2

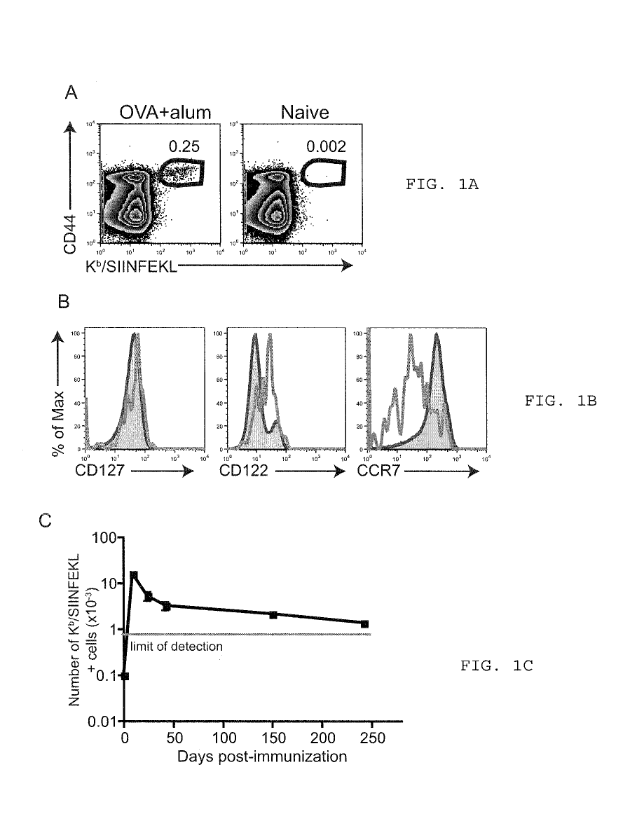

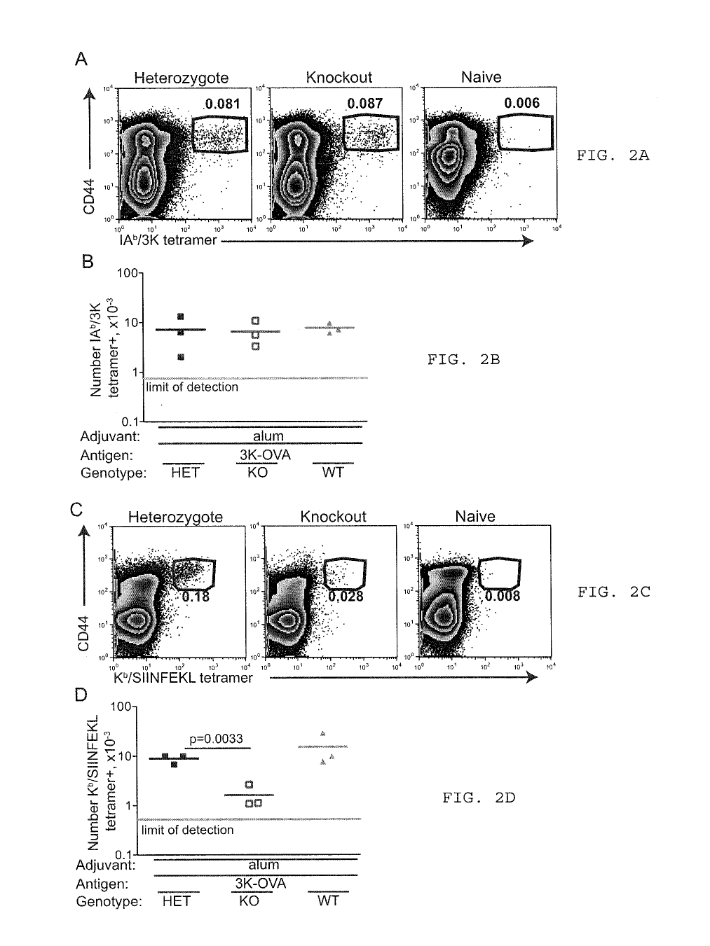

This example illustrates that protein is delivered with alum adjuvant primes CD8 T cells. The number and phenotype of antigen specific CD8 T cells activated following immunization of C57BL/6 (B6) with ovalbumin (OVA) protein and alum were determined using K^{b} MHC class I tetramers containing the peptide SIINFEKL (SEQ ID NO:1) to detect the antigen specific cells by flow cytometry. A clear population of activated cells was found in the spleen 8 days after immunization (FIG. 1A). The memory cells generated from immunization with OVA+ alum expressed markers associated with effector memory cells (FIG. 1B), and these cells formed a fairly stable population of cells that could be detected at least 200 days after immunization (FIG. 1C). Like other CD8 memory T cells (Surh, C. D., and Sprent, J. 2008. Immunity 29:848-862), the antigen specific memory cells underwent homeostatic proliferation to maintain their numbers.

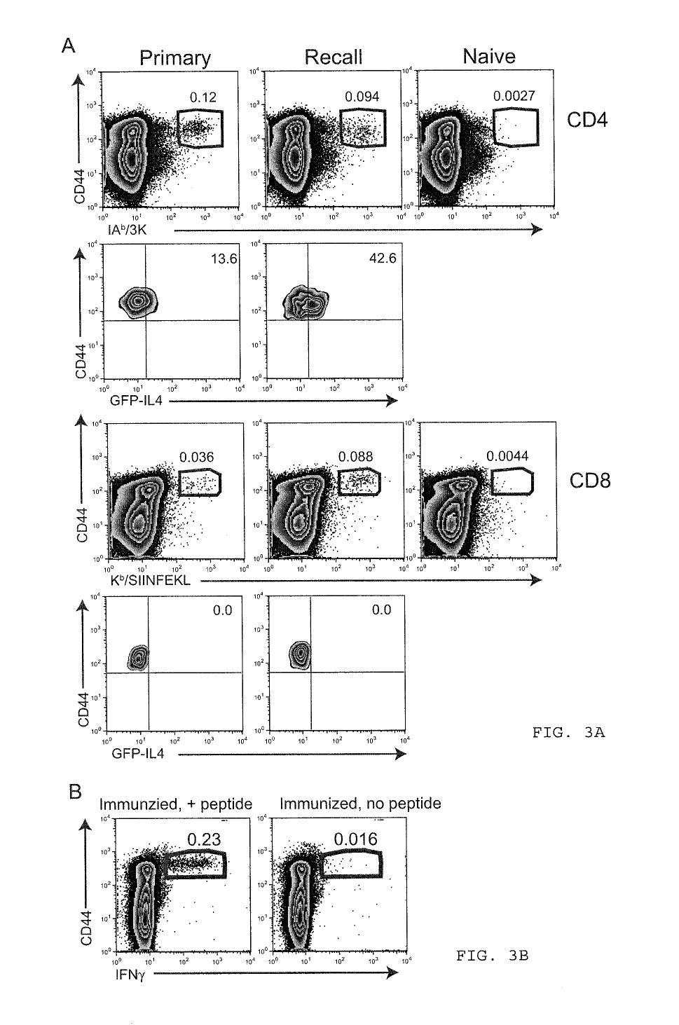

The priming of CD8 T cells by exogenous antigens occurs by a process called cross-presentation that is thought to be mediated by CD8α+CD11c+ dendritic cells (DCs) (Allan, R. S., et al. 2003. Science 301:1925-1928; Bevan, M. J. 1976. J Exp Med 143:1283-1288). Mice deficient in the transcription factor Batf3 lack CD8α+DC and are defective in cross-presentation (Hildner, K., et al. 2008. Science 322:1097-1100). To test whether the presentation of exogenous antigen delivered with alum occurs through a similar process, Batf3 knockout (KO) mice were immunized with OVA conjugated to the CD4 epitope 3K allowing for examination of a CD4 and a CD8 T cell response in the same animals as those having MHC class II tetramers containing the 3K peptide (McKee, A. S., et al. 2009. J Immunol 183:4403-4414). The number of IA^{b}/3K specific CD4 T cells was the same in wild-type and knockout mice, demonstrating that the Batf3 KO mice could respond to protein delivered with alum (FIG. 2A, B). The number of K^{b}/SIINFEKL (SEQ ID NO:1) tetramer+ cells was, however, barely above background in the knockout mice (FIG. 2C, D) demonstrating that cross-priming after antigen delivery with alum occurs via a similar cross-presentation pathway as described for viral or tumor challenge (Hildner, K., et al. 2008. Science 322:1097-1100).

### Example 3

This example illustrates that alum induces a type 2 response in activated CD4 T cells but a type 1 response in activated CD8 T cells.

To demonstrate that the CD8 T cells activated with antigen+ alum made a Th2 response, antigen specific cells in immunized IL4 mRNA reporter mice (4Get) in which cells that express message for the IL4 gene co-express GFP (30) were examined. For a positive control antigen specific CD4 T cells in the same animals again using OVA protein that had been conjugated to 3K peptide were examined. Some of the IA^{b}/3K tetramer+CD4 T cells were GFP+, however no GFP+K^{b}/SIINFEKL (SEQ ID NO:1)+ cells were detected (FIG. 3A). In contrast antigen specific CD8 IFNγ producing cells after immunization with OVA+ alum (FIG. 3B) were detected. Thus although CD4 T cells make a Th2 response to antigens injected with alum, CD8 T cells do not and instead make a type 1 immune response.

### Example 4

This example illustrates that antigen delivered with alum provides partial protection from influenza virus.

For determining whether CD8 T cells primed with antigen+ alum could protect mice from a viral infection, an epitope from the nucleoprotein of influenza A, NP₃₆₆₋₇₄(NP), was coupled to a protein (OVA, or bovine serum albumin, BSA). With is approach only CD8 T cells, and not CD4 T cells or B cells, were primed against viral antigens, yet CD4 T cell help, in the shape of CD4 T cells specific for OVA or BSA, was available. Thus, the effects of the immunization on the course of a subsequent influenza virus infection must be due to the primed CD8 T cells. Responses to NP peptide conjugated to either OVA or BSA was determined to demonstrate that the cross-presentation of the epitope did not require the presence of OVA. Regardless of which protein was used, consistently the same results have been determined.

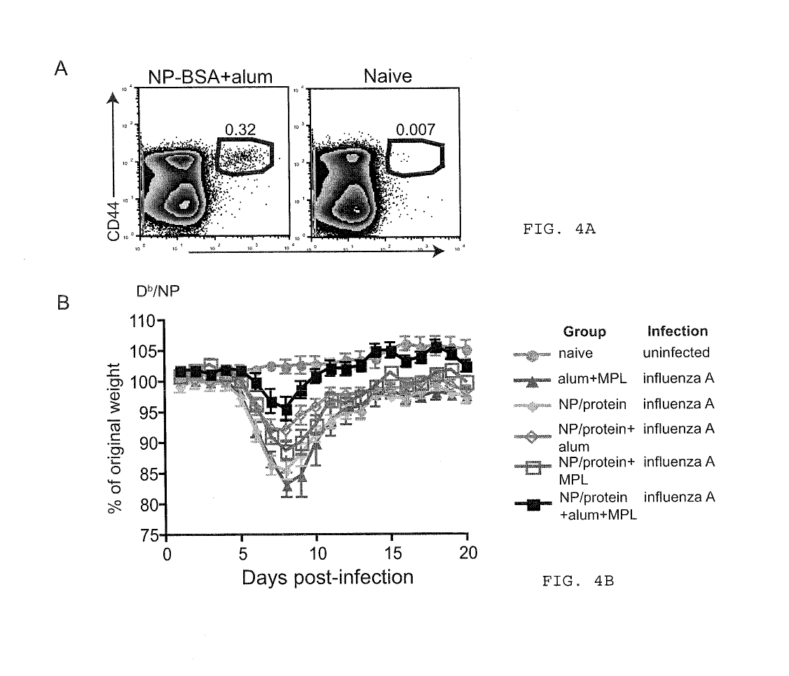

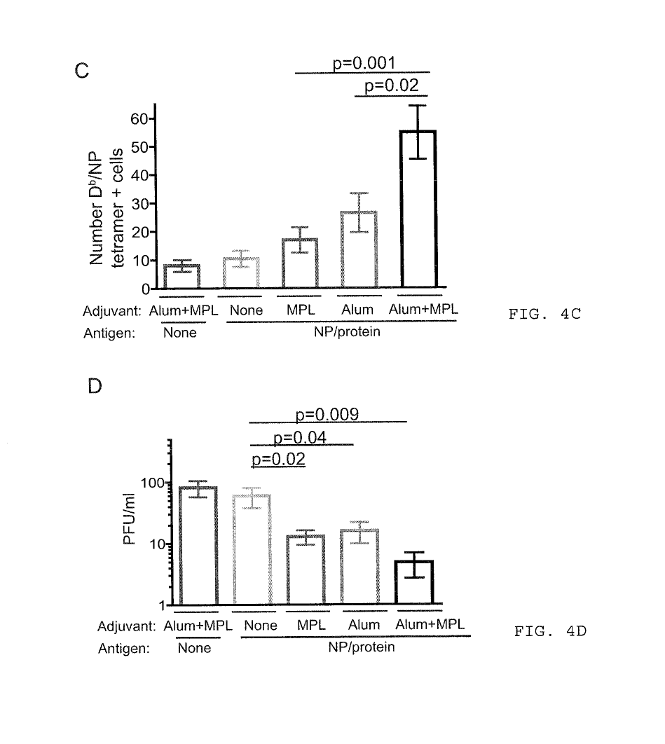

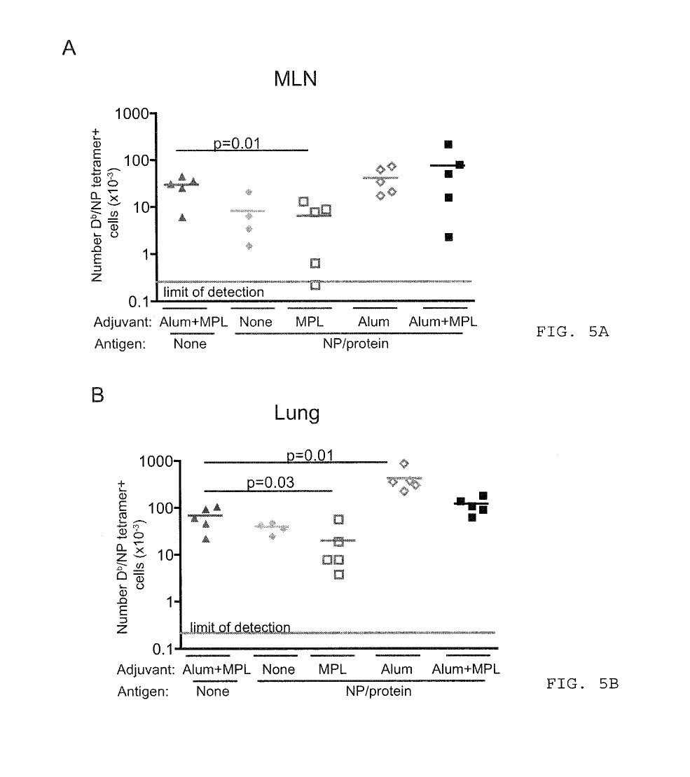

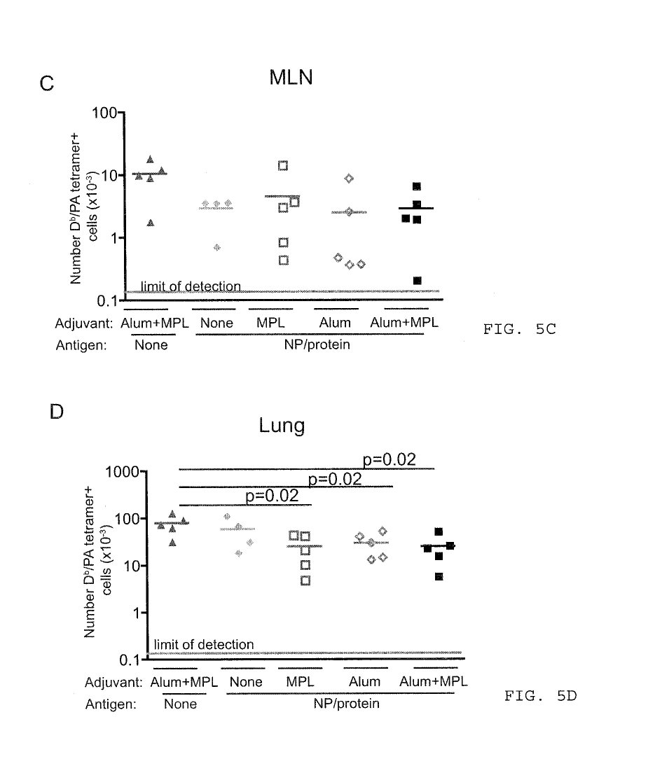

CD8 NP specific cells were confirmed to be primed by staining spleen cells from mice immunized 9 days previously with NP/protein+ alum with D^{b}/NP tetramers (FIG. 4A). These cells were determined to protect mice from influenza virus challenge. The mice were immunized with NP/protein+ alum and then along with control mice, the mice were infected intranasally with influenza A 5-14 weeks after immunization. Mice previously injected with adjuvant alone or immunized with soluble NP/protein lost a significant amount of weight over the first 8 days, then slowly regained some of this weight over the next 12 days. Mice primed with NP/protein+ alum, lost less weight (p=0.05) (FIG. 4B). This protection was, however, not complete and the mice did not return to their original starting weight by day 20 after infection.

### Example 5

This example illustrates that the addition of MPL improves CD8 T cell-mediated protection from influenza.

Mice primed with NP/protein and alum+ MPL lost significantly less weight following infection than did those primed with NP/protein+ alum alone (p=0.0002) and very quickly regained their starting weight (FIG. 4B). Mice primed with antigen delivered with MPL alone lost more weight than those given the combination of both adjuvants (p=0.003) and did not regain their starting weight by the end of the experiment. Therefore, the alum and MPL combination acted in a synergistic fashion to enhance the protective functions of the memory cells.

Four days after infection, it was determined that NP specific CD8 T cells in the lungs of mice that had been primed with NP/protein delivered with one or both adjuvants, with the largest number of cells found in mice given both adjuvants (FIG. 4C). This early recruitment of memory cells to the lungs correlated with a reduction in viral titers, which were most significantly reduced in mice immunized with NP/protein with both adjuvants (FIG. 4D).

In all animals there was a similar CD8 T cell response to the D^{b}/NP epitope in the draining mediastinal lymph node (MLN) 8 days after infection, although some of the mice immunized with NP/protein+MPL contained very few D^{b}/NP tetramer+ cells (FIG. 5A). A similar pattern was seen in the lung although the mice immunized with NP/protein+ alum did contain a greater number of cells (FIG. 5B).

The response to a second immunodominant influenza A epitope, PA₂₂₄₋₂₃₃ was determined. PA specific CD8 T cells were not visible on day 4 but could be clearly identified on day 8. All the mice had similar numbers of cells present in both the lymph node (FIG. 5C) and the lung on day 8 (FIG. 5D). Animals primed with either of the adjuvants or the combination did have slightly fewer PA specific cells probably reflecting the reduction of virus in these mice at day 4. Therefore, although the mice immunized with NP/protein and both adjuvants had reduced disease, this did not prevent a primary response to other viral epitopes.

### Example 6

This example illustrates that MPL enhances cytotoxic differentiation of alum-primed CD8 T cells.

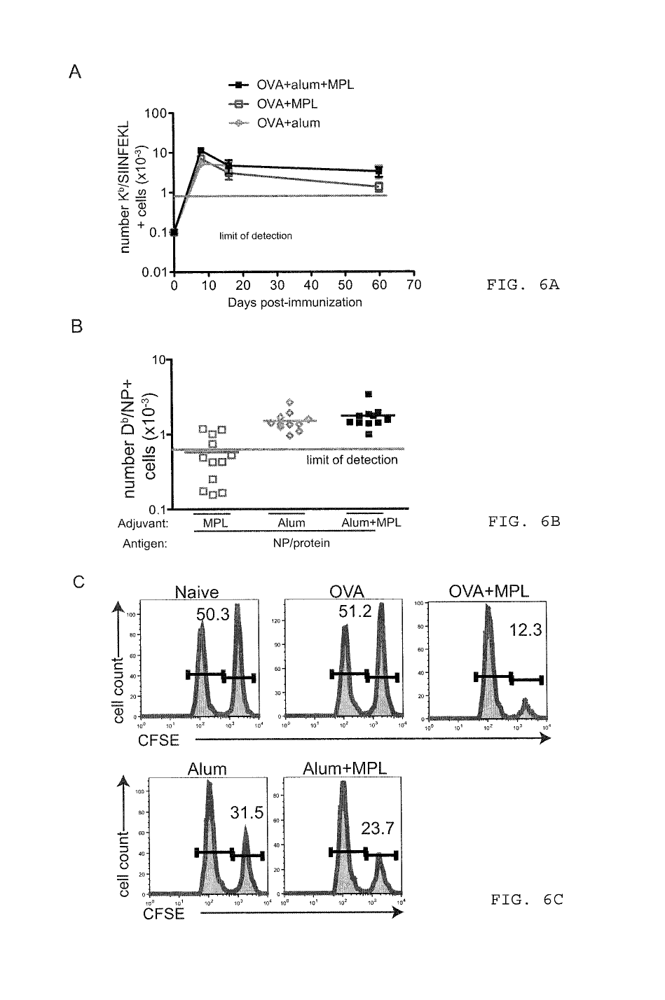

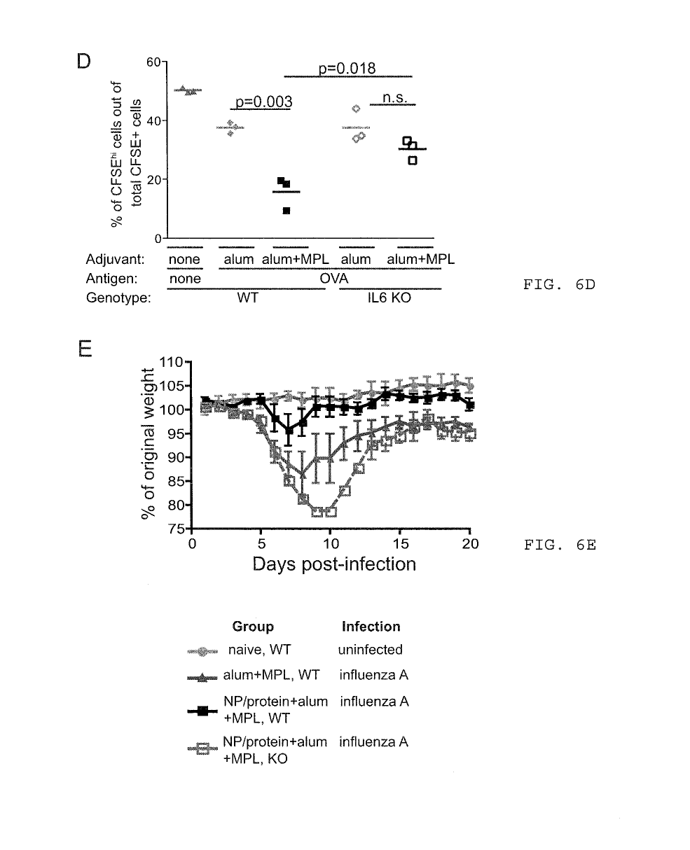

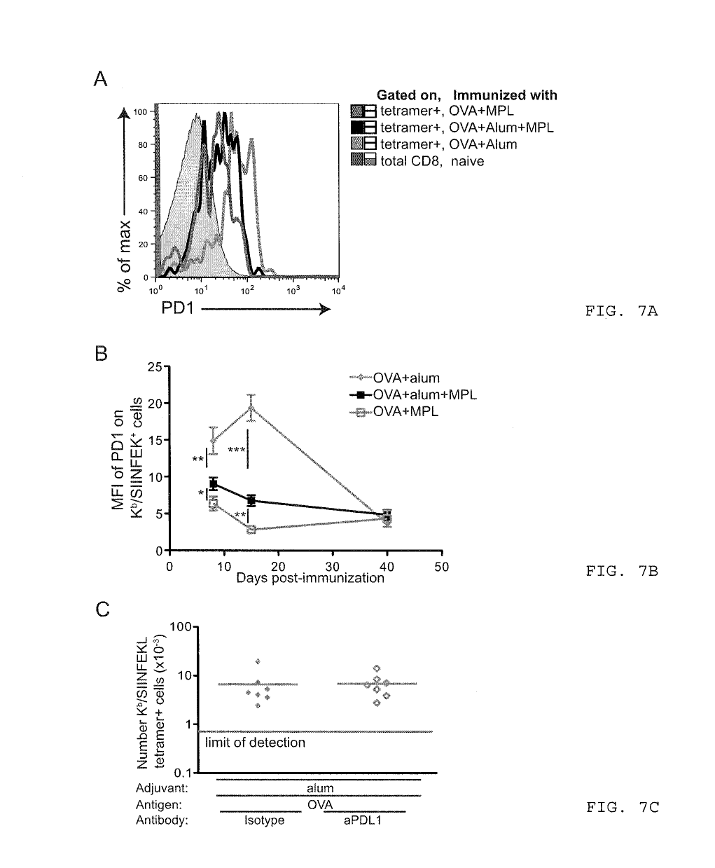

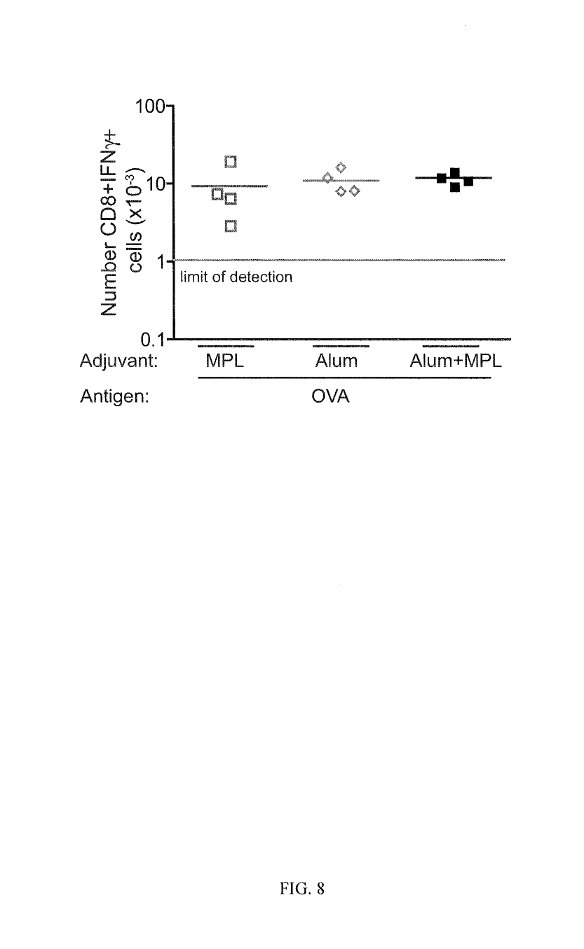

The combination of adjuvants was determined to be required for the best protection by analysis of the number and effector response of the CD8 T cells. Early after immunization, the numbers of antigen specific T cells were similar in mice immunized with OVA with either or both adjuvants (FIG. 6A), and the numbers of IFNγ producing cells were also similar (FIG. 8). However, the number of specific cells markedly declined in mice immunized with OVA+MPL (FIG. 6A), and the number of NP specific cells dropped below detection levels several weeks after immunization in mice primed with NP/protein+MPL (FIG. 6B). Such numbers declined less markedly in animals given antigen+ alum or the combination of adjuvants. The lack of protection found in mice primed with protein+MPL only was due to the poor generation of long-lived memory cells.

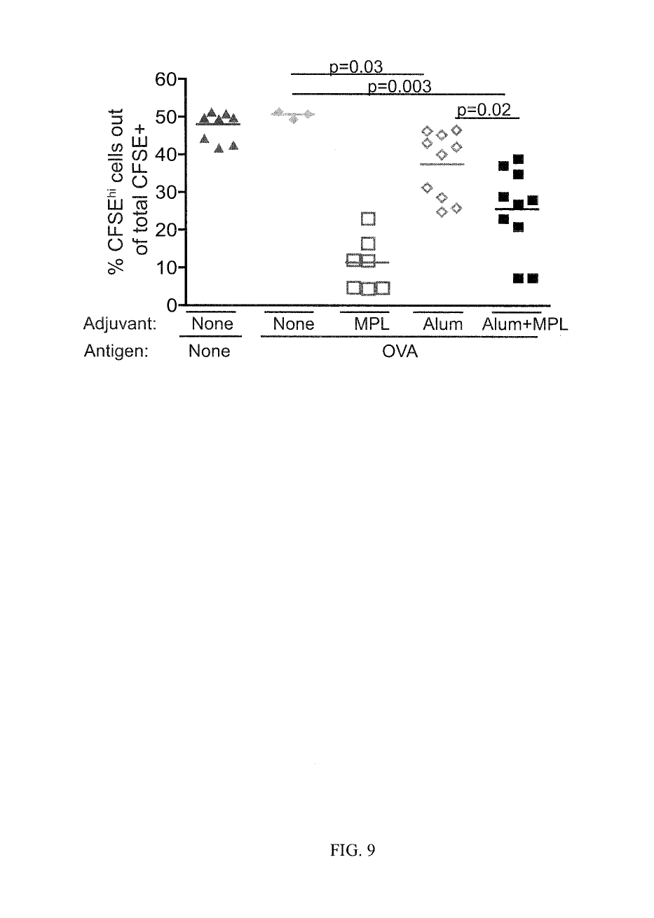

The number of memory cells generated in response to protein+ alum or alum+MPL was similar (FIG. 6A and FIG. 6B). Alum-generated memory cells were determined to be less effective at protecting mice from challenge (FIG. 4B), as determined by the ability of the primed cells to kill target cells in vivo. Immunized mice were injected with peptide-pulsed and non-pulsed splenocytes that could be distinguished since they had been labeled with different intensities of the fluorescent dye, carboxyfluorescein succinimidyl ester (CFSE). T cells primed with OVA+MPL killed the peptide-pulsed target cells very efficiently. The addition of alum reduced this killing to some extent, but priming with OVA+ alum alone generated CD8 T cells with only limited cytotoxic activity (FIG. 6C and FIG. 9).

IL6 was determined to be required for the enhanced cytotoxic response in the presence of MPL by immunizing wild-type and IL6 knockout mice with OVA delivered with alum or alum+ MPL. There was no difference in the number of K^{b}/SIINFEKL (SEQ ID NO:1) tetramer+ in wild-type or knockout mice immunized with alum or alum+ MPL. While there was no difference in the cytotoxic response in mice primed with OVA+ alum, the increased killing observed in wild-type mice given both adjuvants was not found in the IL6 knockout mice (FIG. 6D). Therefore, MPL induced IL6 was essential for the efficient differentiation of cytotoxic CD8 T cells.

The ability to kill infected cells is required for protection. Perforin knockout mice were primed with NP/protein+ alum+ MPL and then infected with influenza A. The wild-type immunized mice were protected, however, the perforin knockout mice lost significant amounts of weight and 4 out 10 had to be sacrificed due to excessive weight loss compared to none in the immunized wild-type group (FIG. 6E).

### Example 7

This example illustrates that increased PD1 expression on alum-primed CD8 T cells inhibits granzyme B expression and specific killing.

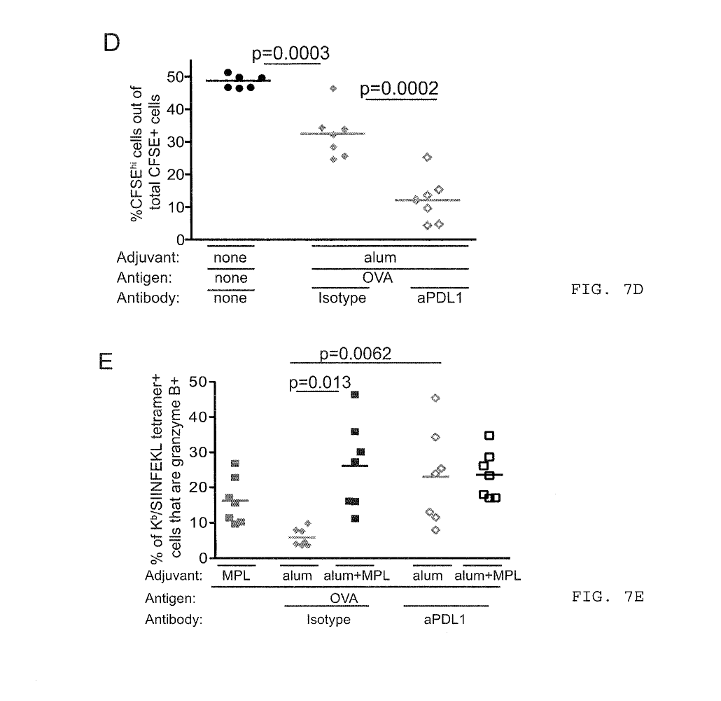

Alum-primed CD8 T cells were determined to be poor cytotoxic cells due to the T cells expressing inhibitory molecules. T cells activated in the presence of alum expressed higher levels PD1 (FIG. 7A). Co-injection of alum with MPL reduced the expression of PD 1 on the antigen specific T cells and mice immunized with antigen+MPL had lower levels still (FIG. 7A and FIG. 7B). In all cases, PD1 expression declined as the activated cells differentiated into memory cells (FIG. 7B).

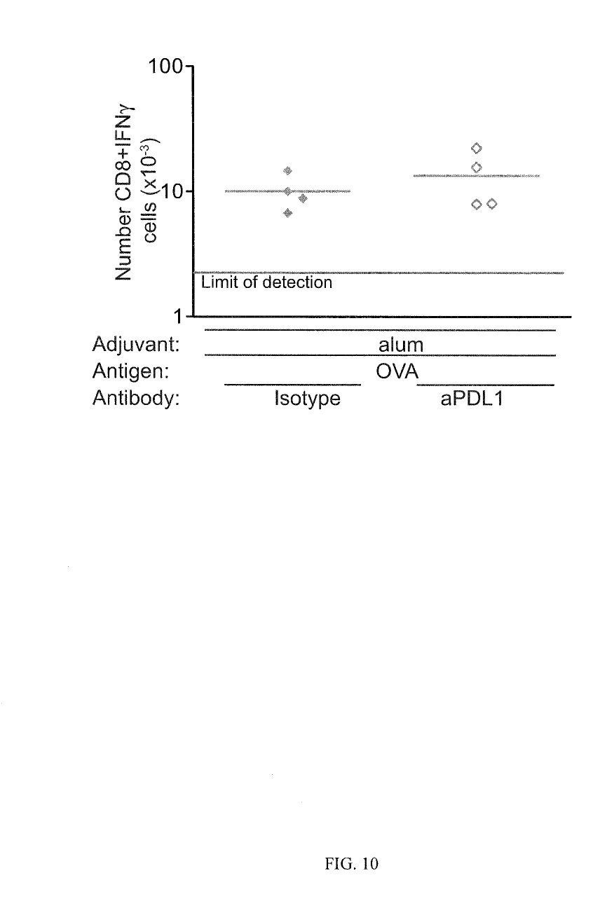

The high expression of PD1 on the alum-primed CD8 T cells was determined to inhibit their responses. Immunized mice were treated with either an isotype control antibody or anti-PDL1. The number of K^{b}/SIINFEKL (SEQ ID NO:1) tetramer+ T cells primed by immunization with OVA+ alum was unaffected by the presence of the anti-PDL1 antibody (FIG. 7C) as was the production of IFNγ by these cells (FIG. 10). However, T cells primed in mice treated with anti-PDL1 could kill target cells more effectively (FIG. 7D). Thus the high PD1 expression on the alum-primed T cells inhibited their ability to kill target cells.

The expression of granzyme B, the molecule required to set of the apoptotic pathway inside target cells by the activated CD8 T cells was determined. Few K^{b}/SIINFEKL (SEQ ID NO:1) tetramer+ T cells from mice primed with OVA+ alum expressed granzyme B ex vivo, in contrast either co-immunization with MPL or treatment with anti-PDL1 resulted in an increase in the percent of antigen specific T cells that expressed granzyme B (FIG. 7E). Together these data show that T cells primed in the presence of alum upregulate PD1 expression and that this inhibits their differentiation into CTLs. The presence of MPL reduces the expression of PD1 allowing increased CTL differentiation that can be mimicked by the presence of the anti-PDL1 antibody that blocks the interaction between PD1 and PDL1.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

Each publication and reference cited herein is incorporated herein by reference in its entirety.

### REFERENCES

1. Harty, J. T., Tvinnereim, A. R., and White, D. W. 2000. CD8+ T cell effector mechanisms in resistance to infection. Annu Rev Immunol 18:275-308.
2. Plotkin, S. A. 2008. Vaccines: correlates of vaccine-induced immunity. Clin Infect Dis 47:401-409.
3. Webster, R. G., Bean, W. J., Gorman, O. T., Chambers, T. M., and Kawaoka, Y. 1992. Evolution and ecology of influenza A viruses. Microbiol Rev 56:152-179.
4. Boon, A. C., de Mutsert, G., van Baarle, D., Smith, D. J., Lapedes, A. S., Fouchier, R. A., Sintnicolaas, K., Osterhaus, A. D., and Rimmelzwaan, G. F. 2004. Recognition of homo- and heterosubtypic variants of influenza A viruses by human CD8+ T lymphocytes. J Immunol 172:2453-2460.
5. Braciale, T. J. 1977. Immunologic recognition of influenza virus-infected cells. II. Expression of influenza A matrix protein on the infected cell surface and its role in recognition by cross-reactive cytotoxic T cells. J Exp Med 146:673-689.
6. Jameson, J., Cruz, J., Terajima, M., and Ennis, F. A. 1999. Human CD8+ and CD4+ T lymphocyte memory to influenza A viruses of swine and avian species. J Immunol 162:7578-7583.
7. Kees, U., and Krammer, P. H. 1984. Most influenza A virus-specific memory cytotoxic T lymphocytes react with antigenic epitopes associated with internal virus determinants. J Exp Med 159:365-377.
8. Townsend, A. R., McMichael, A. J., Carter, N. P., Huddleston, J. A., and Brownlee, G. G. 1984. Cytotoxic T cell recognition of the influenza nucleoprotein and hemagglutinin expressed in transfected mouse L cells. Cell 39:13-25.
9. Yewdell, J. W., Bennink, J. R., Smith, G. L., and Moss, B. 1985. Influenza A virus nucleoprotein is a major target antigen for cross-reactive anti-influenza A virus cytotoxic T lymphocytes. Proc Natl Acad Sci USA 82:1785-1789.
10. Christensen, J. P., Doherty, P. C., Branum, K. C., and Riberdy, J. M. 2000. Profound protection against respiratory challenge with a lethal H7N7 influenza A virus by increasing the magnitude of CD8(+) T-cell memory. J Virol 74:11690-11696.
11. Epstein, S. L. 2006. Prior H1N1 influenza infection and susceptibility of Cleveland Family Study participants during the H2N2 pandemic of 1957: an experiment of nature. J Infect Dis 193:49-53.
12. Furuya, Y., Chan, J., Regner, M., Lobigs, M., Koskinen, A., Kok, T., Manavis, J., Li, P., Mullbacher, A., and Alsharifi, M. 2010. Cytotoxic T cells are the predominant players providing cross-protective immunity induced by {gamma}-irradiated influenza A viruses. J Virol. 84 (9) 4212.
13. McMichael, A. J., Gotch, F. M., Noble, G. R., and Beare, P. A. 1983. Cytotoxic T-cell immunity to influenza. N Engl J Med 309:13-17.
14. O'Neill, E., Krauss, S. L., Riberdy, J. M., Webster, R. G., and Woodland, D. L. 2000. Heterologous protection against lethal A/HongKong/156/97 (H5N1) influenza virus infection in C57BL/6 mice. J Gen Virol 81:2689-2696.
15. Ulmer, J. B., Donnelly, J. J., Parker, S. E., Rhodes, G. H., Felgner, P. L., Dwarki, V. J., Gromkowski, S. H., Deck, R. R., DeWitt, C. M., Friedman, A., et al. 1993. Heterologous protection against influenza by injection of DNA encoding a viral protein. Science 259:1745-1749.
16. Appay, V., Douek, D. C., and Price, D. A. 2008. CD8+ T cell efficacy in vaccination and disease. Nat Med 14:623-628.
17. Doherty, P. C., and Kelso, A. 2008. Toward a broadly protective influenza vaccine. J Clin Invest 118:3273-3275.
18. McKee, A. S., Macleod, M. K., Kappler, J. W., and Marrack, P. 2010. Immune mechanisms of protection: can adjuvants rise to the challenge? BMC Biol 8:37.
19. Castellino, F., and Germain, R. N. 2007. Chemokine-guided CD4+ T cell help enhances generation of IL-6RalphahighIL-7Ralpha high prememory CD8+ T cells. J Immunol 178:778-787.
20. Mbow, M. L., De Gregorio, E., Valiante, N. M., and Rappuoli, R. 2010. New adjuvants for human vaccines. Curr Opin Immunol 22:411-416.
21. Newman, M. J., Wu, J. Y., Gardner, B. H., Munroe, K. J., Leombruno, D., Recchia, J., Kensil, C. R., and Coughlin, R. T. 1992. Saponin adjuvant induction of ovalbumin-specific CD8+ cytotoxic T lymphocyte responses. J Immunol 148:2357-2362.
22. Giannini, S. L., Hanon, E., Moris, P., Van Mechelen, M., Morel, S., Dessy, F., Fourneau, M. A., Colau, B., Suzich, J., Losonksy, G., et al. 2006. Enhanced humoral and memory B cellular immunity using HPV16/18 L1 VLP vaccine formulated with the MPL/aluminium salt combination (ASO4) compared to aluminium salt only. Vaccine 24:5937-5949.
23. Vandepapeliere, P., Horsmans, Y., Moris, P., Van Mechelen, M., Janssens, M., Koutsoukos, M., Van Belle, P., Clement, F., Hanon, E., Wettendorff, M., et al. 2008. Vaccine adjuvant systems containing monophosphoryl lipid A and QS21 induce strong and persistent humoral and T cell responses against hepatitis B surface antigen in healthy adult volunteers. Vaccine 26:1375-1386.
24. McKee, A. S., Munks, M. W., MacLeod, M. K., Fleenor, C. J., Van Rooijen, N., Kappler, J. W., and Marrack, P. 2009. Alum induces innate immune responses through macrophage and mast cell sensors, but these sensors are not required for alum to act as an adjuvant for specific immunity. J Immunol 183:4403-4414.
25. Surh, C. D., and Sprent, J. 2008. Homeostasis of naive and memory T cells. Immunity 29:848-862.
26. Allan, R. S., Smith, C. M., Belz, G. T., van Lint, A. L., Wakim, L. M., Heath, W. R., and Carbone, F. R. 2003. Epidermal viral immunity induced by CD8alpha+ dendritic cells but not by Langerhans cells. Science 301:1925-1928.
27. Bevan, M. J. 1976. Cross-priming for a secondary cytotoxic response to minor H antigens with H-2 congenic cells which do not cross-react in the cytotoxic assay. J Exp Med 143:1283-1288.
28. Hildner, K., Edelson, B. T., Purtha, W. E., Diamond, M., Matsushita, H., Kohyama, M., Calderon, B., Schraml, B. U., Unanue, E. R., Diamond, M. S., et al. 2008. Batf3 deficiency reveals a critical role for CD8alpha+ dendritic cells in cytotoxic T cell immunity. Science 322:1097-1100.
29. Marrack, P., McKee, A. S., and Munks, M. W. 2009. Towards an understanding of the adjuvant action of aluminium. Nat Rev Immunol 9:287-293.
30. Mohrs, M., Shinkai, K., Mohrs, K., and Locksley, R. M. 2001. Analysis of type 2 immunity in vivo with a bicistronic IL-4 reporter. Immunity 15:303-311.
31. Didierlaurent, A. M., Morel, S., Lockman, L., Giannini, S. L., Bisteau, M., Carlsen, H., Kielland, A., Vosters, O., Vanderheyde, N., Schiavetti, F., et al. 2009. ASO4, an aluminum salt- and TLR4 agonist-based adjuvant system, induces a transient localized innate immune response leading to enhanced adaptive immunity. J Immunol 183:6186-6197.
32. Renauld, J. C., Vink, A., and Van Snick, J. 1989. Accessory signals in murine cytolytic T cell responses. Dual requirement for IL-1 and IL-6. J Immunol 143:1894-1898.
33. Smyth, M. J., Ortaldo, J. R., Bere, W., Yagita, H., Okumura, K., and Young, H. A. 1990. IL-2 and IL-6 synergize to augment the pore-forming protein gene expression and cytotoxic potential of human peripheral blood T cells. J Immunol 145:1159-1166.
34. Galandrini, R., Cernetti, C., Albi, N., Dembech, C., Terenzi, A., Grignani, F., and Velardi, A. 1991. Interleukin-6 is constitutively produced by human CTL clones and is required to maintain their cytolytic function. Cell Immunol 138:11-23.
35. Ming, J. E., Steinman, R. M., and Granelli-Piperno, A. 1992. IL-6 enhances the generation of cytolytic T lymphocytes in the allogeneic mixed leucocyte reaction. Clin Exp Immunol 89:148-153.
36. Topham, D. J., Tripp, R. A., and Doherty, P. C. 1997. CD8+ T cells clear influenza virus by perforin or Fas-dependent processes. J Immunol 159:5197-5200.
37. Francisco, L. M., Sage, P. T., and Sharpe, A. H. 2010. The PD-1 pathway in tolerance and autoimmunity. Immunol Rev 236:219-242.
38. Brown, K. E., Freeman, G. J., Wherry, E. J., and Sharpe, A. H. 2010. Role of PD-1 in regulating acute infections. Curr Opin Immunol 22:397-401.
39. Barber, D. L., Wherry, E. J., Masopust, D., Zhu, B., Allison, J. P., Sharpe, A. H., Freeman, G. J., and Ahmed, R. 2006. Restoring function in exhausted CD8 T cells during chronic viral infection. Nature 439:682-687.
40. Yewdell, J. W. 2010. Designing CD8+ T cell vaccines: it's not rocket science (yet). Curr Opin Immunol 22:402-410.
41. Shen, L., and Rock, K. L. 2006. Priming of T cells by exogenous antigen cross-presented on MHC class I molecules. Curr Opin Immunol 18:85-91.
42. Lin, M. L., Zhan, Y., Proietto, A. I., Prato, S., Wu, L., Heath, W. R., Villadangos, J. A., and Lew, A. M. 2008. Selective suicide of cross-presenting CD8+ dendritic cells by cytochrome c injection shows functional heterogeneity within this subset. Proc Natl Acad Sci USA 105:3029-3034.
43. Hornung, V., Bauernfeind, F., Halle, A., Samstad, E. O., Kono, H., Rock, K. L., Fitzgerald, K. A., and Latz, E. 2008. Silica crystals and aluminum salts activate the NALP3 inflammasome through phagosomal destabilization. Nat Immunol 9:847-856.
44. Eisenbarth, S. C., Colegio, O. R., O'Connor, W., Sutterwala, F. S., and Flavell, R. A. 2008. Crucial role for the Nalp3 inflammasome in the immunostimulatory properties of aluminium adjuvants. Nature 453:1122-1126.
45. Li, H., Willingham, S. B., Ting, J. P., and Re, F. 2008. Cutting edge: inflammasome activation by alum and alum's adjuvant effect are mediated by NLRP3. J Immunol 181:17-21.
46. Franchi, L., and Nunez, G. 2008. The Nlrp3 inflammasome is critical for aluminium hydroxide-mediated IL-1beta secretion but dispensable for adjuvant activity. Eur J Immunol 38:2085-2089.
47. Peixoto, A., Evaristo, C., Munitic, I., Monteiro, M., Charbit, A., Rocha, B., and Veiga-Fernandes, H. 2007. CD8 single-cell gene coexpression reveals three different effector types present at distinct phases of the immune response. J Exp Med 204:1193-1205.
48. Kool, M., Soullie, T., van Nimwegen, M., Willart, M. A., Muskens, F., Jung, S., Hoogsteden, H. C., Hammad, H., and Lambrecht, B. N. 2008. Alum adjuvant boosts adaptive immunity by inducing uric acid and activating inflammatory dendritic cells. J Exp Med.
49. Wang, M., Lamberth, K., Harndahl, M., Roder, G., Stryhn, A., Larsen, M. V., Nielsen, M., Lundegaard, C., Tang, S. T., Dziegiel, M. H., et al. 2007. CTL epitopes for influenza A including the H5N1 bird flu; genome-, pathogen-, and HLA-wide screening. Vaccine 25:2823-2831.
50. Heiny, A. T., Miotto, O., Srinivasan, K. N., Khan, A. M., Zhang, G. L., Brusic, V., Tan, T. W., and August, J. T. 2007. Evolutionarily conserved protein sequences of influenza a viruses, avian and human, as vaccine targets. PLoS One 2:e1190.
51. Kreijtz, J. H., de Mutsert, G., van Baalen, C. A., Fouchier, R. A., Osterhaus, A. D., and Rimmelzwaan, G. F. 2008. Cross-recognition of avian H5N1 influenza virus by human cytotoxic T-lymphocyte populations directed to human influenza A virus. J Virol 82:5161-5166.
52. Lee, L. Y., Ha do, L. A., Simmons, C., de Jong, M. D., Chau, N. V., Schumacher, R., Peng, Y. C., McMichael, A. J., Farrar, J. J., Smith, G. L., et al. 2008. Memory T cells established by seasonal human influenza A infection cross-react with avian influenza A (H5N1) in healthy individuals. J Clin Invest 118:3478-3490.
53. Ng, W. C., Gilbertson, B., Lim, B., Zeng, W., Jackson, D. C., and Brown, L. E. 2009. Lipopeptide vaccines illustrate the potential role of subtype-crossreactive T cells in the control of highly virulent influenza. Influenza Other Respi Viruses 3:177-182.
54. Poon, L. L., Leung, Y. H., Nicholls, J. M., Perera, P. Y., Lichy, J. H., Yamamoto, M., Waldmann, T. A., Peiris, J. S., and Perera, L. P. 2009. Vaccinia virus-based multivalent H5N1 avian influenza vaccines adjuvanted with IL-15 confer sterile cross-clade protection in mice. J Immunol 182:3063-3071.
55. Wang, J., Oberley-Deegan, R., Wang, S., Nikrad, M., Funk, C. J., Hartshorn, K. L., and Mason, R. J. 2009. Differentiated human alveolar type II cells secrete antiviral IL-29 (IFN-lambda 1) in response to influenza A infection. J Immunol 182:1296-1304.
56. Crawford, F., Kozono, H., White, J., Marrack, P., and Kappler, J. 1998. Detection of antigen-specific T cells with multivalent soluble class II MHC covalent peptide complexes. Immunity 8:675-682.
57. Lenz, D. C., Kurz, S. K., Lemmens, E., Schoenberger, S. P., Sprent, J., Oldstone, M. B., and Homann, D. 2004. IL-7 regulates basal homeostatic proliferation of antiviral CD4+ T cell memory. Proc Natl Acad Sci USA 101:9357-9362.

1. A composition comprising an isolated internal pathogenic protein, a Toll-like receptor (TLR) agonist and an aluminum salt.

2. The composition of claim 1, wherein the isolated internal pathogenic protein is selected from the group consisting of influenza nucleoprotein (NP), matrix protein 1 (M1), matrix protein 2 (M2), non-structural-1 (NS1), non-structrual-2 (NS2), polymerase acidic (PA), polymerase basic 1 (PB 1), polymerase basic 1 F2 (PB2-F2), and polymerase basic 2 (PB2).

3. The composition of claim 1, wherein the isolated internal pathogenic protein is NP.

4. The composition of claim 3, wherein the NP is from influenza A.

5. The composition of claim 1, wherein the isolated internal pathogenic protein is from a pathogen selected from the group consisting of a virus, parasite and bacteria.

6. The composition of claim 1, wherein the TLR agonist is selected from the group consisting of TLR1, TLR2, TLR3, TLR4, TLR5, TLR6, TLR7, TLR8, TLR9, TLR10, TLR11, TLR12, and TLR13.

7. The composition of claim 6, wherein the TLR agonist is selected from the group consisting of a lipopolysaccharide (LPS) derivative or mimetic, monophosphoryl lipid A (MPL) or RC529.

8. The composition of claim 7, wherein the LPS derivative is MPL.

9. The composition of claim 1, wherein the aluminum salt is selected from the group consisting of alum, potassium aluminum sulfate, aluminum phosphate, and aluminum hydroxide.

10. The composition of claim 9, wherein the aluminum salt is alum.

11. The composition of claim 1, wherein the composition may be administered to a subject orally, subcutaneously, intramuscularly, intravenously, by aerosol to the respiratory tract, or intradermally.

12. A composition comprising an influenza A nucleoprotein, MPL and alum.

13. A method for protecting a subject against infection by a pathogen comprising administering to the subject a composition comprising an isolated internal pathogenic protein, a Toll-like receptor (TLR) agonist and an aluminum salt.

14. The method of claim 13, wherein the pathogen is capable of causing a disease selected from the group consisting of influenza, a rhinovirus associated disease, adenovirus associated disease, malaria and Listeria infection.

15. The method of claim 14, wherein the influenza is selected from the group consisting of influenza A, influenza B and influenza C.

16. The method of claim 13, wherein the isolated internal pathogenic protein is selected from the group consisting of influenza nucleoprotein (NP), M1, M2, NS1, NS2, PA, PB1, PB1-F2, and PB2.

17. The method of claim 16, wherein the isolated internal pathogenic protein is NP.

18. The method of claim 17, wherein the NP is from influenza A.

19. The method of claim 13, wherein the TLR agonist is selected from the group consisting of TLR1, TLR2, TLR3, TLR4, TLR5, TLR6, TLR7, TLR8, TLR9, TLR10, TLR11, TLR12, and TLR13.

20. The method of claim 19, wherein the TLR agonist is selected from the group consisting of a lipopolysaccharide (LPS) derivative or mimetic, MPL or RC529.

21. The method of claim 20, wherein the LPS derivative is MPL.

22. The method of claim 13, wherein the aluminum salt is selected from the group consisting of alum, potassium aluminum sulfate, aluminum phosphate, and aluminum hydroxide.

23. The method of claim 22, wherein the aluminum salt is alum.

24. The method of claim 13, wherein the route of administration may be intra-peritoneal, oral, subcutaneous, intramuscular, intravenous, by aerosol to the respiratory tract, or intradermal.

25. A method for protecting a subject against infection by influenza comprising administering to the subject a composition comprising an influenza A nucleoprotein, MPL and alum.

26. The method of claim 13, wherein the subject is human.
